(19) United States
(12) Patent Application Publication
(10) Pub. No.: US 2025/0152148 A1
(43) Pub. Date: May 15, 2025

(54) DEVICES, KITS, AND METHODS FOR SAMPLES

(71) Applicant: Care For There LLC, Carlsbad, CA (US)

(72) Inventors: Thomas B. Moyles, Carlsbad, CA (US); Rajasingam S. Jeyendran, Carlsbad, CA (US)

(21) Appl. No.: 18/946,746

(22) Filed: Nov. 13, 2024

Related U.S. Application Data
(60) Provisional application No. 63/599,523, filed on Nov. 15, 2023.

(51) Int. Cl. A61B 10/00

(52) U.S. Cl. CPC A61B 10/0096; A61B 10/0058

(57) ABSTRACT

Devices, kits, and methods for samples, including devices, kits, and methods for the collection, transport, and/or testing of semen samples are described herein. A kit for a sample is provided, which includes a sample device having a body, a first cap configured to removably couple to the body at a first open end, and a second cap configured to removably couple to the body at a second open end. A cap can include an opening and a removable plug to seal the opening and/or a seal that receives a pipette and automatically seals when the pipette is removed. The kit can include a stand for holding one or more sample devices and/or a preservative suitable for preserving the sample at room temperature for between 1-120 hours or more.

### CROSS-REFERENCE TO RELATED APPLICATIONS

This application claims priority to U.S. Provisional Patent Application Ser. No. 63/599,523, filed Nov. 15, 2023. This and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

### FIELD OF THE INVENTION

The present disclosure relates generally to devices, kits, and methods for samples, including devices, kits, and methods for the collection, transport, and/or testing of semen samples.

### BACKGROUND

A semen analysis or test typically involves looking at the volume or quality of someone's sperm to, for example, detect fertility issues, and/or determine whether a vasectomy was successful. The collection and analysis of samples, especially semen samples, can be awkward for the person providing the sample, sometimes referred to as a subject, and challenging for a person extracting and/or analyzing the sample, sometimes referred to as a technician.

Typically a specimen must be collected such that only the semen sample is collected in a sample cup and a lid is screwed onto the cup. Upon arrival at the lab, a technician unscrews the lid from the cup in order to extract a sample from the cup (e.g., using a pipette), and applies it to a slide for analysis. The collection, transport, and/or analysis of semen samples can be quite time consuming, and can require numerous steps on the part of the subject and technician, among others. There are opportunities for error at various steps.

### SUMMARY

Accordingly, devices, kits, and methods are provided herein that can advantageously enable a person or subject the ability to collect and safely transport a sample (or have the semen sample transported) for analysis in a device. In some aspects, a lab can conduct an analysis, in whole or in part, using the same sample device,

In an aspect, a kit for a sample is provided, including a sample device. In some embodiments, the device has a body having a first open end and a second open end, a first cap configured to removably couple to the body at the first open end; and a second cap configured to removably couple to the body at the second open end. In some embodiments, at least one of the first and second caps comprises a first opening sized and dimensioned for receiving at least a portion of a pipette, tube (e.g., air pump tube), or other object. In some embodiments, a removable plug sized and dimensioned to seal the first opening can be provided. The plug can be made of any suitable material or materials, including, for example, a plastic, a rubber, a foam, a wood, a glass, a silicone, an acrylic, or a cork. In some embodiments, a seal that is configured to receive at least a portion of a pipette, tube, or other object (e.g., having a slit opening), and self-seal when the object is removed can be provided. In some embodiments, a seal that is configured to be pierceable by a pipette, tube, syringe, etc. can be provided.

Each cap can removably couple to the body via any suitable mechanism. For example, a cap can be a screw cap, screw cap with O-ring, snap cap, a plug cap, flanged plug cap, pierceable cap, or any other suitable cap. In some embodiments, a cap comprises a first end portion and a first sealing portion that extends from the first end portion. The sealing portion can comprise a ring shape that is sized and dimensioned to be securely received by an open end of the body. For example, the sealing portion can have an outer diameter that is the same as or slight smaller than (e.g., less than 5%, less than 10% smaller than) an inner diameter of the open end. In some embodiments the first end portion can comprise a pull tab or other component that a user can utilize to remove the cap from the tube.

In some embodiments, the kit comprises a preservative suitable for preserving the sample (e.g., semen sample) for a period of at least 1 hour, at least 2 hours, at least 3 hours, at least 5 hours, at least 8 hours, at least 12 hours, at least 18 hours, at least 24 hours, at least 1 day, at least 2 days, at least 3 days, at least 4 days, at least 5 days, or any other suitable amount of time at room temperature. In some embodiments, the kit comprises a preservative suitable for preserving the sample (e.g., semen sample) for a period of at least 1 hour, at least 2 hours, at least 3 hours, at least 5 hours, at least 8 hours, at least 12 hours, at least 18 hours, at least 24 hours, at least 1 day, at least 2 days, at least 3 days, at least 4 days, at least 5 days, or any other suitable amount of time at room temperature when the sample is contained in the sample device as described herein.

In some embodiments, the kit comprises a stand for one or more sample devices. The stand can comprise a base portion, a holder portion comprising at least one holder sized and dimensioned to hold at least one sample device of the one or more sample devices such that the at least one sample device is positioned above the base, and a support portion extending between the base portion and the holder portion. In some embodiments, the base and the holder portions extend about perpendicularly relative to the support portion. For example, an angle between the base portion and the support portion can be between 75-105 degrees, and an angle between the holder portion and the support portion can be between 75-105 degrees.

In some embodiments, the sample device further comprises a first coupling component, and wherein the at least one holder comprises a second coupling component complementary to the first coupling component. In some embodiments, at least one of the sample device and the holder portion (e.g., holder) of the stand comprises a gripping material. In some embodiments, the at least one holder is sized and dimensioned to hold the sample device such that the sample device does not contact the base. In some embodiments, the at least one holder comprises a C-shaped portion extending away from the support portion. In some embodiments, the C-shaped portion comprises end portions such that a distance between the end portions widen to receive and/or hold a portion of a sample device, and automatically (e.g., with flexible end portions) or manually (e.g., via an adjustment mechanism) narrow when the portion of the sample device is removed. In some embodiments, the C-shaped portion comprises a gripping material that helps maintain a sample device in place (e.g., when contacting the body portion and/or a gripping material on the body portion). In some embodiments, the C-shaped portion comprises a compressible material to help maintain a sample device in place and/or protect the sample device.

In an aspect, a sample device is provided, comprising a body having a first open end and a second open end, a first cap configured to couple to the body at the first open end, wherein the first cap comprises a central opening, a second cap configured to couple to the body at the second open end, and a plug configured to seal the central opening.

In an aspect, a method of providing a sample for analysis is provided, comprising depositing a sample into a condom comprising a top portion and a reservoir portion, placing the condom including the sample in a collection tube having a first end by securing a top portion of the condom around the first end of the collection tube with the reservoir portion positioned within the tube, and securing the top portion of the condom around the first end of the collection tube with a top lid.

Other advantages and benefits of the disclosed kits, devices, systems and methods will be apparent to one of ordinary skill with a review of the following description.

### BRIEF DESCRIPTION OF THE DRAWINGS

The details of embodiments of the present disclosure, both as to their structure and operation, can be gleaned in part by study of the accompanying drawings, in which like reference numerals refer to like parts, and in which:

FIG. 1 illustrates a kit comprising a stand, a sample device, and a preservative, according to an embodiment;

FIG. 2A illustrates a sample device with a condom partially secured to the device, according to an embodiment;

FIG. 2B illustrates the sample device of FIG. 2A with the condom of FIG. 2A secured to the device;

FIG. 3. is a flowchart illustrating a method, according to an embodiment;

FIG. 4 is a flowchart illustrating another method, according to another embodiment;

FIG. 5A illustrates a sample device of a kit with both caps removed, according to an embodiment; and

FIG. 5B illustrates a stand of the kit, according to an embodiment.

### DETAILED DESCRIPTION

The detailed description set forth below, in connection with the accompanying drawings, is intended as a description of various embodiments and is not intended to represent the only embodiments in which the disclosure may be practiced. The detailed description includes specific details for the purpose of providing a thorough understanding of the embodiments. However, it will be apparent that those skilled in the art will be able to understand the disclosure without these specific details. In some instances, well-known structures and components are shown in simplified form for brevity of description. Some of the surfaces have been left out or exaggerated for clarity and ease of explanation.

The present disclosure is directed to devices, kits, and methods for samples, including devices, kits, and methods for the collection, transport, extraction and/or testing of semen samples.

In some aspects, a sample device is provided that allows for safely transporting the sample, while also enabling the lab to utilize a pre-packaged sample that is already set up for at least one of extracting the sample and analyzing the sample, thereby eliminating one or more steps and saving significant preparation time.

In some aspects, a sample device is provided, comprising a body (e.g., an elongated body) having a first open end, and a first cap configured to couple to the elongated body at the first open end, for example, to seal the first open end of the elongated body.

In some embodiments, the elongated body comprises a second open end opposite the first open end.

In some embodiments, a second cap is provided and configured to couple to the elongated body at the second open end, for example, to seal the second open end of the elongated body.

In some embodiments, the first cap comprises an opening, for example, a central opening, and a seal or plug configured to seal or plug the central opening. In some embodiments, the central opening is sized and dimensioned for receiving at least a portion of a pipette, which allows for removing or extracting a sample from within the sample device without removing the first cap. In some embodiments, the central opening is sized and dimensioned for receiving at least a portion of an air pump tube (or tube of any device that can be used to deliver air, for example, to expand a condom therein).

In some embodiments, the elongated body comprises a tube with a circular cross-section. In some embodiments, the first open end and the second open end each have a circular shape. In some embodiments, the elongated body has a consistent diameter along its length. In some embodiments, the elongated body has a varying diameter along its length.

In some embodiments, the first cap comprises a first end portion and a first sealing portion that extends from the first end portion. In some embodiments, the first sealing portion comprises a ring shape and is sized and dimensioned to be securely received by the first open end of the elongated body.

In some embodiments, the first end portion of the first cap comprises a pull tab. In some embodiments, the second cap comprises a second end portion and a second sealing portion that extends from the second end portion. In some embodiments, the second sealing portion comprises a ring shape and is sized and dimensioned to be securely received by the second open end of the elongated body. In some embodiments, the second end portion comprises a pull tab. In some embodiments, the plug comprises a handle portion (e.g., any portion that allows a user to readily remove the plug from cap). In some embodiments, the elongated body is made from a plastic such as a clear plastic.

In another aspect, a stand that is provided is configured to securely hold one or more sample devices. The stand can be configured to securely hold one or more sample devices in any suitable configuration, including an upright configuration where a top cap is positioned directly above a bottom cap.

In some embodiments, a stand for one or more sample devices is provided, comprising a base portion (e.g., a portion that contacts a lab table), a holder portion comprising at least one holder sized and dimensioned to hold at least one sample device of the one or more sample devices such that the at least one sample device is positioned above the base, and a support portion extending between the base portion and the holder portion. In some embodiments, the at least one sample device comprises a first coupling component and the holder portion comprises a second coupling component complementary to the first coupling component.

While all suitable coupling components are contemplated herein, a few example coupling components include snap-fit interlocking components (e.g., cantilever snap, torsion snap, and/or annular snap joints). In some embodiments, the first coupling component comprises an annular protrusion of and/or coupled to the elongated body, and the second coupling component comprises an upper portion of the holder and/or the holder as a whole acting as a block to the annular protrusion. In some embodiments, the sample device has a varying diameter, for example, including a wider portion, and the holder and wider portion prevents the sample device from dropping below a predetermined area relative to the holder.

In some embodiments, the at least one holder is sized and dimensioned to hold the at least one sample device such that the at least one sample device does not contact the base (there is a space between the sample device and the base). The space can be, for example, at least 0.5 inch, at least 1 inch, at least 2 inches, at least 3 inches, between 0.5-5 inches, between 3-5 inches, at least 5 inches, or any other suitable distance between a bottom of the sample device and a top surface of the base. In some embodiments, the at least one holder comprises a C-shaped portion (whether or not it is curved, angled, etc.) extending about perpendicularly from the support portion. In some embodiments, the at least one holder comprises an O-shaped portion (whether or not it is curved, angled, etc.) extending about perpendicularly from the support portion. In some embodiments, the C-shaped portion comprises flexible end portions. In some embodiments, the C-shaped portion comprises a gripping material (e.g., a silicone, rubber, foam rubber, a gel). In some embodiments, the C-shaped portion comprises a compressible material (e.g., a foam, a gel, a rubber). In some embodiments, the C-shaped portion is configured to receive the at least one sample device via a snap fit. In some embodiments, the C-shaped portion is adjustable (e.g., has an adjustable diameter, has a C-shaped portion with arms that could be adjusted to reduce or increase a space between the arms.

In another aspect, a kit is provided comprising one or more containers (e.g., vials) of a preservative for preserving one or more samples, and one or more sample devices as described herein. The kit can optionally comprise a stand configured to securely hold one or more sample devices and/or containers configured to hold the preservatives. In some aspects, the kit can include a condom suitable for use when obtaining/providing a semen sample for analysis, for example, to detect fertility issues, and/or determine whether a vasectomy was successful.

In another aspect, a kit is provided comprising at least one sample device (any of the sample device(s) described herein), and at least one stand (any of the stand(s) described herein). In some embodiments, the at least one sample device comprises between 2-100, between 2-50, between 2-25, between 2-10, at least 1, at least 2, at least 3, at least 4, at least 5, or any other suitable number of sample devices. In some embodiments, the kit further comprises a preservative (e.g., a one or more containers of a preservative to preserve the sample during any stages between collection and analysis, for example, during delivery, mail, transport).

Contemplated preservatives include, for example, formaldehyde fixatives, and/or fixatives described in U.S. Pat. No. 5,618,664 to Ann A. Kiessling, which is incorporated herein in its entirety. Contemplated preservatives also include, for example, curcumin formulations (e.g., curcumin powder). Adamczak A, Ożarowski M, Karpiński TM. Curcumin, a Natural Antimicrobial Agent with Strain-Specific Activity. Pharmaceuticals (Basel). 2020 Jul. 16; 13(7):153. doi: 10.3390/ph13070153. PMID: 32708619; PMCID: PMC7408453, which is incorporated herein in its entirety, describes curcumin as a potential antibacterial agent, but with a very selective activity. Applicant surprisingly discovered curcumin can be used for as a preservative for sperm samples. In some embodiments, the kit further comprises one or more pipettes. In some embodiments, the kit further comprises one or more air pump devices and/or systems.

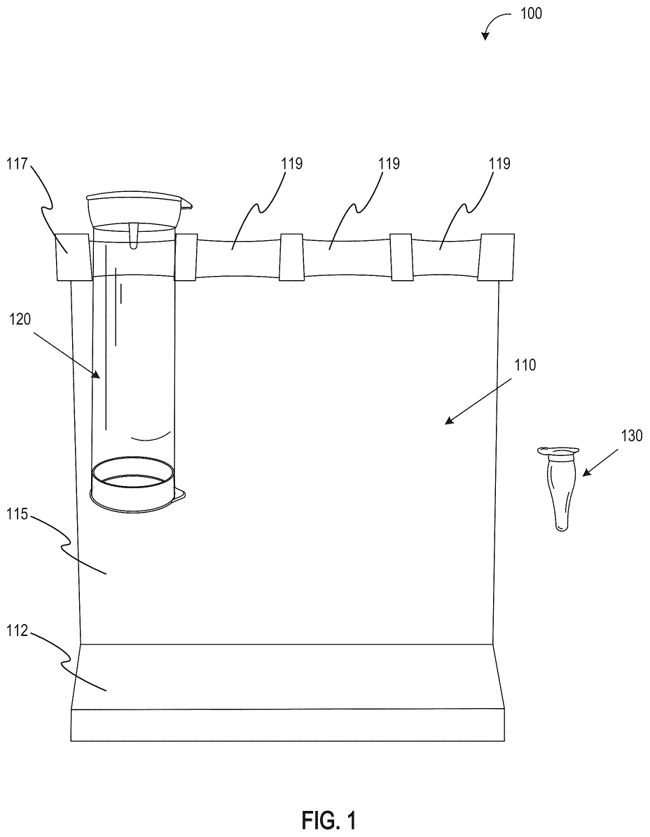

Referring now to FIG. 1, a kit 100 comprising a stand 110, a sample device 120, and a preservative 130 is illustrated. Stand 100 comprises a base portion 112, a support portion 115, and a holder portion 117 having at least one holder 119 (here, at least 4 with 3 full holders shown). Sample device 120 comprises an elongated body having a first open end and a second open end, a first cap secured to the first open end, and a second cap secured to the second open end. First cap (top cap) includes a central opening that is shown with a plug secured in the central opening. Each of the first cap and the second cap (bottom cap) include a pull tab that a user can pull to remove the cap from the elongated body. It should be appreciated that while the pull tab is shown extending from a side of an end wall or end portion, it is also contemplated the pull tab can be positioned extending from a top of the end wall or end portion of the cap, or any other suitable location. For example, the pull tab could comprise a knob or handle positioned above the end wall or end portion of the cap.

Preservative 130 comprises a curcumin powder that is secured in a vial with a removable or partially removable top. It should be appreciated that multiple sample devices and/or multiple preservatives can be provided in a kit.

While the disclosure herein generally refers to sample devices comprising an elongated body, it should be appreciated that in some embodiments, the body may not be elongated. For example, the body may comprise a spherical shape, or a tube shape where a length from a first end to a second end (e.g., top to bottom length) is not greater or significantly greater than a side to side length or diameter. The sample device can comprise a body of any suitable size and shape, and have any suitable cross sectional shape. In some embodiments, the elongated body only comprises one open end and one cap, with an opposite end being closed or sealed. In some embodiments, the elongated body has a top to bottom length of between 1-10, between 2-8, between 5-10 inches, between 1-8 inches, between 1-5 inches, or between 3-6 inches. In some embodiments, the elongated body has a width or diameter of between 0.5-3 inches, between 0.5-1.5 inches, between 0.5-1.25 inches, between 0.5-2.5 inches, or between 0.8-2 inches.

In some embodiments, the body has a circular cross-section. However, other shapes are contemplated, for example, a body with an oval cross-section, a rectangular cross-section, a hexagonal cross-section, an octagonal cross-section, or any other suitable shape. The body and each cap can be of any suitable material or materials. In some embodiments, each of the body and the cap(s) are made from the same material(s). In some embodiments, the body and the cap(s) are made from different material(s). In some embodiments, the body is made of a plastic and/or a glass. In some embodiments, the cap(s) are made from a plastic, a cork, and/or a rubber.

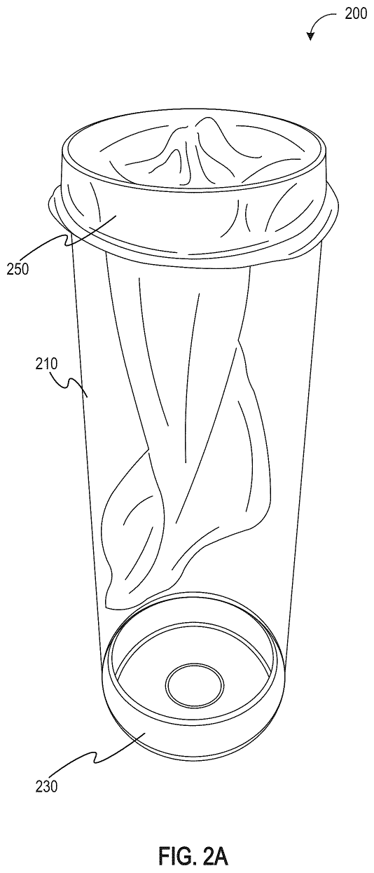

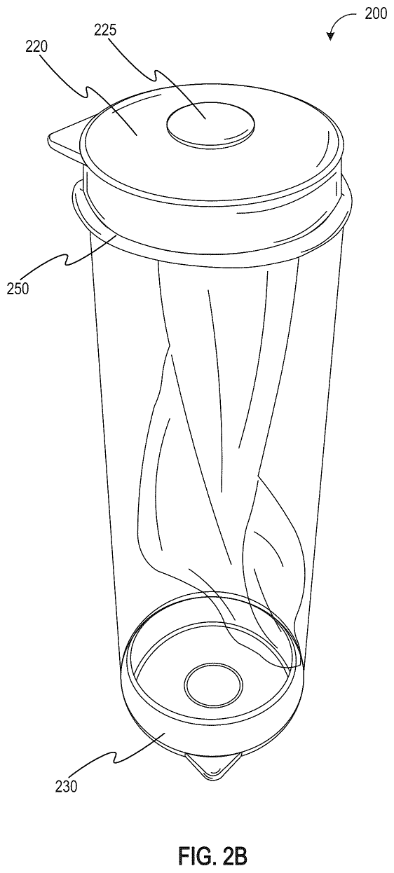

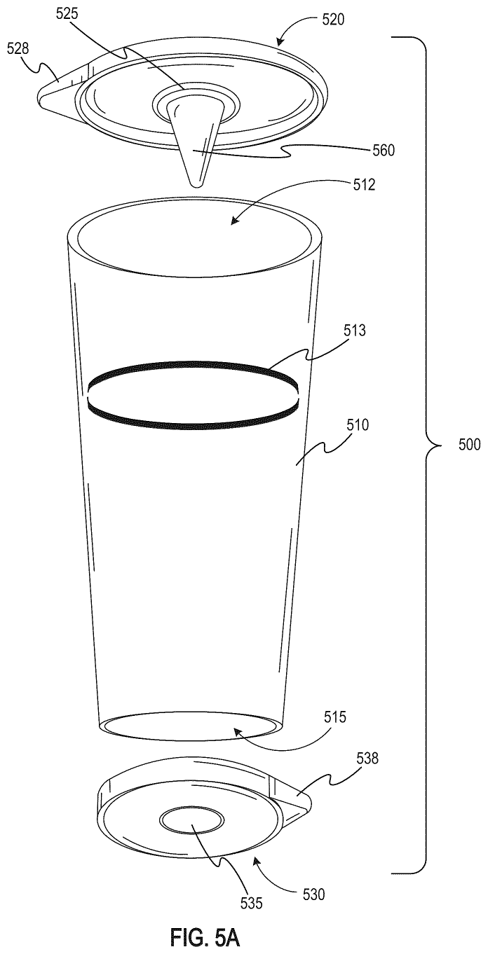

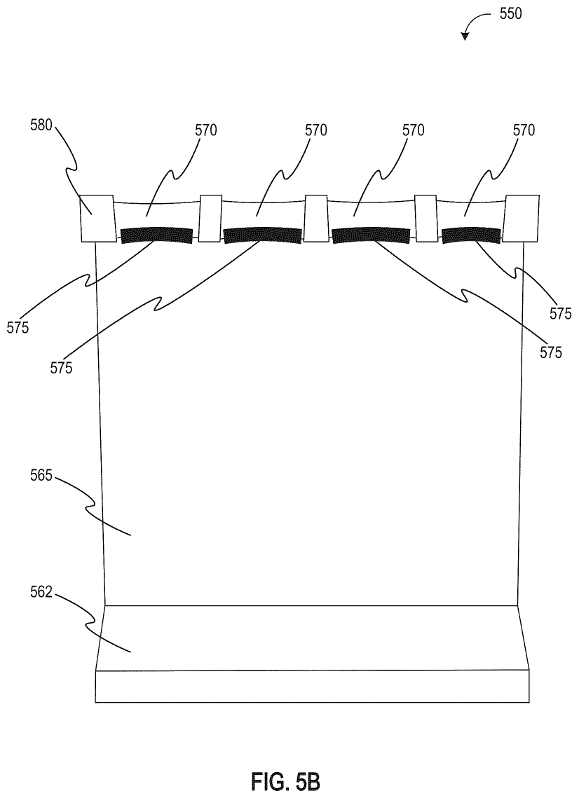

One or more of the caps of a sample device can comprise an opening, for example, a central or non-central hole having a diameter of between 3-10 mm, or about 6 mm, or any other suitable size or shape. The opening can advantageously be sized and dimensioned to receive a portion of a transfer pipette or other pipette such that the pipette tip can be inserted into the condom positioned in the sample device to extract some or all of the sample. The opening can be plugged via a plug (e.g., as illustrated in FIG. 1, FIGS. 2B, and 5A). The plug can comprise any suitable material and be of any suitable size and shape. For example, some contemplated plugs can comprise a rubber, plastic and/or cork plug having an end portion (or handle portion) and a sealing portion. The end portion can be larger than the opening such that it remains outside of the sample device when the plug is secured to the opening. The sealing portion can comprise an elongated piece that is wider at a first end (adjacent end portion/handle portion) and gradually gets narrower from the first end to the second end. The sealing portion or a portion thereof can be compressible and have a diameter that is slightly larger than the opening when not compressed, and the same as or slightly smaller than the opening when compressed. Additionally or alternatively, the opening can comprise a compressible material, for example, lining the opening. In some embodiments, the plug can be fully removable from the cap. In some embodiments, the plug can be connected to the cap such that upon removal from the opening, the plug is still connected to the cap (so it is not lost). For example, the plug can be coupled to the cap via a connector. Additionally or alternatively, the cap can be coupled to the body via a connector.

The sample devices described herein can advantageously allow a subject to simply collect a sample, and transport it or cause it to be transported to a laboratory (e.g., by mailing) or other location in a device such that the sample can be simply and efficiently collected/extracted from the sample device. In some embodiments, the sample is transported in a device such that is already prepped for analysis when it arrives in the laboratory or other location. No other known solution exists that allows the patient to transport their sample (e.g., to a laboratory) in the same condom the sample was collected in (e.g., ejaculated into). When using a sample device of the disclosure according to some embodiments, it is contemplated that a patient does not need to empty the sample from the condom into a sample cup, which reduces the risk of error and makes the process more convenient and comfortable. Further, use of the sample devices of the disclosure can advantageously improve the speed of collection and extraction and/or analysis for both the subject and the technician.

Now turning to FIGS. 2A-2B, a sample device 200 is shown with a top cap removed (FIG. 2A), and with a top cap secured to an elongated body and ready for transport (FIG. 2B). Sample device 200 comprises an elongated body 210 having a first open end and a second open end. Top cap 220 comprises a plug 225, and is configured to couple to the first open end (as shown in FIG. 2B). Bottom cap 230 is configured to couple to the second open end (as shown in FIGS. 2A and 2B). In FIG. 2A, a reservoir portion of the condom is positioned within the elongated body, and the top end portion of the condom 250 is extended over the first open end of elongated body 210. In FIG. 2B, top cap 220 is secured to first open end of elongated body 210 with some or all of the top end portion of condom 250 sandwiched between end wall and/or inner surface of side portion of top cap 220 and an open end and/or outer surface of elongated body 210.

Now turning to FIG. 5A, a sample device 500 is shown with first cap 520 removed from body 510, and second cap 530 removed from body 510. Body 510 of sample device 500 is shown with a first open end 512 and second open end 515. However, it should be appreciated that in some embodiments, a sample device only includes one open end and a closed or covered end. Body 510 includes a holder assist component 513, which can be, for example, a coupling component, a gripping material, a compressible material, or any other component that helps hold a sample device in a desired position, for example, on a holder of a stand. The holder assist component 513 can include, for example, a gripping material, a compressible material, a hook or loop fastener portion, or any other material or suitable coupling component. First cap 520 is configured to removably couple to and seal first open end 512 of body 510. In some embodiments, first cap 520 is configured to removably couple to and seal first open 512 of body 510 with a portion of a condom positioned therebetween. First cap 520 comprises an opening 525, a plug 560, and a pull tab 528. Second cap 530 is configured to removably couple to and seal second open end 515 of body 510. Second cap 530 comprises a pull tab 538 and optionally a pierceable portion 535 (e.g., an opening sealed with a pierceable material). It should be appreciated that in some embodiments, the second cap 530 can lack a pierceable portion 535. For example, second cap 530 can lack an opening. As another example, second cap 530 can include an opening without also including a pierceable portion. In some embodiments, second cap 530 can include an opening and a plug that is configured to seal the opening (e.g., a removable plug). It should be appreciated that second cap 530 can be used as a top cap in place of first cap 520 in some embodiments, and/or that first cap 520 can be used as a bottom cap in place of second cap 530 in some embodiments.

FIG. 5B illustrates a stand 550, which can be used to hold sample device 500 and/or one or more other sample devices. Stand 550 includes a base portion 562, a holder portion 580, and a support portion 565 extending between the base portion 562 and holder portion 580. Holder portion 580 includes a set of holders 570. One, some or all of the holders 570 can optionally include a holder assist component 575, which can be, for example, a coupling component, a gripping material, a compressible material, or any other component that helps the holder hold onto a sample device in a desired position. Holder assist component 575 can be, for example, a gripping material, a compressible material, a fastener portion complementary to a fastener portion of holder assist component 513, or any other suitable material or coupling component.

The sample devices, stands, and kits can be used in any suitable manner to collect a sample and extract a sample therefrom.

In some aspects, a method for providing a sample for analysis is provided, comprising some or all of the following steps: depositing a sample into a condom comprising a top portion (including a rim) and a reservoir portion, placing the condom including the sample in a collection tube (e.g., an elongated body) having a first end by securing a top portion of the condom around the first end of the collection tube with the reservoir portion positioned within the tube, and securing the top portion of the condom around the first end of the collection tube with a top lid. In some embodiments, the rim is outside the tube when the top portion of the condom is secured around the first end of the collection tube. In some embodiments, the collection tube and top lid are components of a sample device, and the sample device comprises any of the sample devices contemplated herein.

In some aspects, a method for removing a sample for analysis is provided, comprising some or all of the following steps: placing a sample device on a stand, the sample device comprising an elongated body having a first end and a first cap coupled to the first end, wherein the sample device holds a condom holding the sample, removing a plug from the top cap to expose an opening, keeping the top cap securely in place coupled to the first end, and inserting a portion of a pipette through opening to collect the sample directly from a reservoir portion of the condom positioned within the tube. In some embodiments, the sample device comprises any of the sample devices contemplated herein. In some embodiments, the stand comprises any of the stands contemplated herein. In some embodiments, a stand is not required for removing the sample for analysis. In some embodiments, prior to inserting the portion of the pipette, the method can comprise the steps of placing a tube of an air delivery device through the opening, and delivering air into a condom secured in the sample device via the air delivery device. In some embodiments, the second cap is removed to allow the condom to expand (e.g., to a length greater than the elongated body). In some embodiments, the condom holds the sample for analysis. In some embodiments, contemplated methods can further comprise applying the sample to a slide or other object for analysis. The analysis can include, among other things, checking pH levels, semen volume, sperm concentration, sperm morphology, sperm motility, time to liquefaction, and vitality.

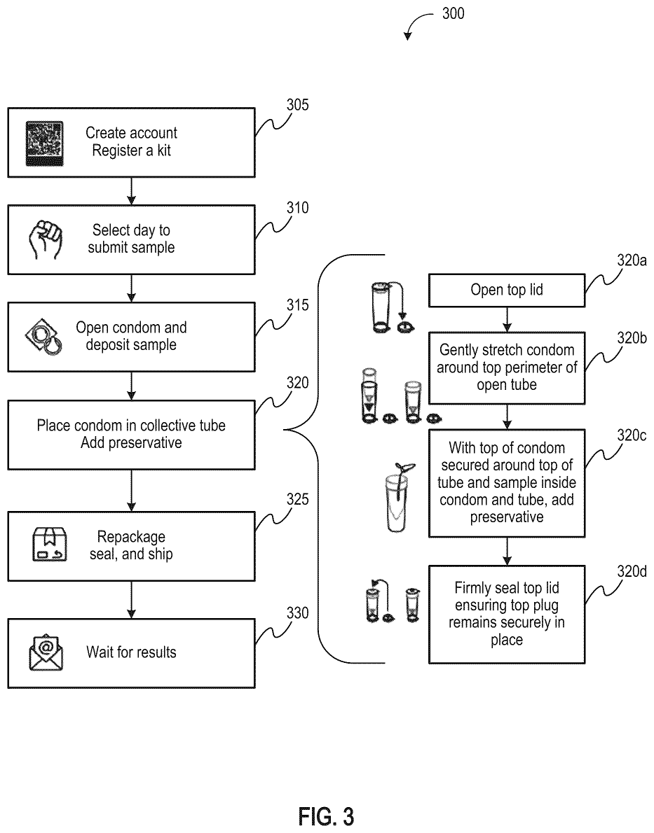

Now turning to FIG. 3, a method 300 from a perspective of a subject is illustrated. It should be appreciated that contemplated methods can comprise some or all of the following steps in any suitable order. It should also be appreciated that contemplated methods can comprise additional steps.

A user may receive a box comprising a sample device and/or kit, which can include the sample device, and optionally a preservative, a shipping label, instructions, and/or a checklist. In some embodiments, the instructions may require a subject to wait at least a minimum amount of time (e.g., 12 weeks) and have a minimum or specific number of ejaculations (e.g., at least 20 ejaculations, 20 ejaculations) after a vasectomy. The box can be used to mail the sample in the sample device to a laboratory or other destination. In step 305, an account is created and a sample device and/or kit is registered. This can be done by visiting a URL included in the device and/or kit, or accessed using a QR code to visit a URL or download an app, among other things.

In step 310, a day is selected to submit a sample (e.g., a semen sample). A sample device and/or kit may include or be accompanied by a checklist. A user can be instructed to, and a user can write the date and time the sample was collected on the checklist and return it with the kit (e.g., in step 325). A user can be instructed to, and a user can ensure they can drop their return box at a mailing location (e.g., UPS store, post office) the same day the sample is collected.

In step 315, a condom is opened and a sample is deposited in the condom. A user can open the condom provided or otherwise obtained, slip it on, and ejaculate into the condom. Once done, the condom can be removed without spilling the sample.

In step 320, the condom is placed in the collection tube. In some embodiments, a preservative (e.g., curcumin) is added to the sample. For example, step 320 can comprise opening a top lid of a sample device (step 320a), gently stretching the condom around a top perimeter (top end portion) of an open tube (elongated body) (step 320b), with the top end of the condom secured around the top perimeter of the open tube, and sample inside the condom and tube, adding a preservative powder (step 320c), and firmly sealing top lid ensuring top plug remains securely in place (step 320d). Note more than one vial of preservative can be provided, for example, with the additional vials provided in case the first vial is spilt.

In step 325, the sample device is repackaged, sealed, and shipped. The collection tube can be placed in the biohazard bag and back in an area for the sample device (e.g., a box area labeled “collection tube””), and the checklist with date and time of collection can be placed in the box. A shipping label (e.g., a pre-paid shipping label) can be placed on the box as instructed (e.g., on the top of the box covering the kit logo), the box can be securely sealed, and dropped off at a UPS location or other mailing location.

In step 330, a user can wait for results until received. In some embodiments, the user receives an email when the kit is received at the destination and/or when the sample has been analyzed and/or when results are ready to be viewed.

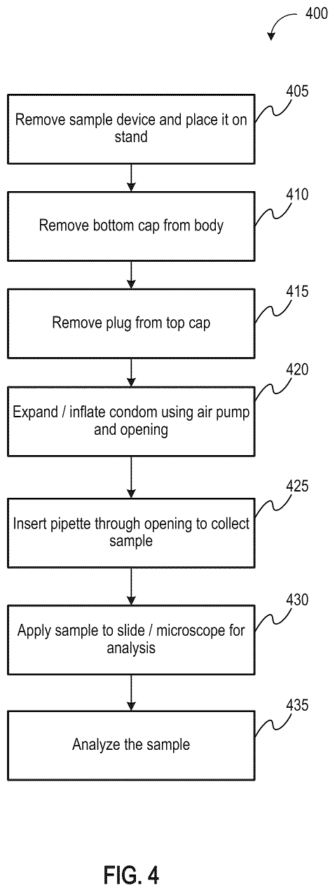

Now turning to FIG. 4, a method 400 from a perspective of a technician or other person extracting and/or analyzing a sample that is collected in a sample device is illustrated. It should be appreciated that contemplated methods can comprise some or all of the following steps in any suitable order. It should also be appreciated that contemplated methods can comprise additional steps.

Once a technician receives a sample device including a sample, the technician can, remove the sample device from the bag/packaging and place it securely in a stand with the top cap including a plug above the bottom cap, in accordance with step 405. It is contemplated that in some embodiments, the bottom of the sample device can be placed on a surface (e.g., a table top) so that the sample can be analyzed. For example, where the body of the sample device has a sufficient length such that a bottom cap is not removed, a technician can place the sample device on a table, and proceed to one or more of the following steps, or any other suitable step(s). Viewed from another perspective, the inventive subject matter can comprise a sample device without a stand and/or other component(s).

In step 410, the technician can remove the bottom cap (which can be a non plugged cap in some embodiments). The condom will remain suspended in place since it will remain securely pinched to the body via the top cap.

In step 415, the technician can remove the plug from the top cap, keeping the top cap securely in place on the body. This will expose the opening (e.g., a 6 mm opening, a 3-10 mm opening, a 3-8 mm opening) in the top cap. Alternatively or additionally, the top cap can comprise a self-sealing component that is configured to seal the opening, but allow a technician to insert a pipette or other component when a pressure is applied. Alternatively or additionally, the top cap can comprise a foil or other pierceable seal over the opening, for example, where the cap is disposed after a single use.

In step 420, using any suitable air pump, for example, a Hygger™ mini air pump, a disposable syringe (e.g., a 10 or 20 mL disposable syringe), or even a turkey baster or similar device, the condom within the body can be slightly expanded/inflated via the opening and the air pump. That will expand the condom enough to make it easier for the technician to extract the sample.

In step 425, the tubing of the air pump can be removed and a technician can insert a standard pipette through the same opening to collect the sample directly from the condom. It is also contemplated that a technician may be able to use the same tube or device to expand/inflate the condom and extract a sample.

In some embodiments, an opening of a sample device cap can comprise a rubber or other piece of material with a slit or other openable component that remains closed until a needle or other component of an air pump is pushed through the slit or other openable component, and wherein the openable component closes back up when the needle or other component is removed. In some embodiments, a cap of a sample device described herein can comprise two or more openings, wherein a first opening allows a user to insert a needle or tube or an air pump, and a second opening allows a user to insert a pipette or other device for extraction of the sample. Each opening can be sealed with a plug, a piece of material with a openable component that is sealed other than when a needle or tube is inserted through the hole, a breakable seal (e.g., a foil seal), or any other suitable component.

In step 430, the technician can apply the sample onto a slide/microscope for analysis. In step 435, the sample can be analyzed. In some embodiments, it is contemplated that some visual analysis can be conducted prior to extracting a sample from the sample device (e.g., where at least a portion of the device (e.g., body) is clear, transparent, or semi-transparent.

Below is a list of some exemplary embodiments of devices, kits, and methods of the inventive subject matter. It should be appreciated that several other embodiments are contemplated herein, and thus the below list is not a complete list of all embodiments contemplated herein.

### Example Embodiments

Embodiment 1. A sample device, comprising: a body having a first open end; and a first cap configured to couple to the elongated body at the first open end.

Embodiment 2. A sample device of embodiment 1, wherein the body comprises an elongated body, and the body comprises a second open end opposite the first open end.

Embodiment 3. The sample device of any of embodiments 1-2, further comprising a second cap configured to couple to the elongated body at the second open end.

Embodiment 4. The sample device of any of embodiments 1-3, wherein the first cap comprises a central opening.

Embodiment 5. The sample device of any of embodiments 1-4, further comprising a plug configured to seal the central opening.

Embodiment 6. The sample device of any of embodiments 1-5, wherein the central opening is sized and dimensioned for receiving at least a portion of a pipette.

Embodiment 7. The sample device of any of embodiments 1-6, wherein the central opening is sized and dimensioned for receiving at least a portion of an air pump tube.

Embodiment 8. The sample device of any of embodiments 1-7, wherein the body comprises a tube with a circular cross-section.

Embodiment 9. The sample device of any of embodiments 1-8, wherein the first open end and the second open end each have a circular shape.

Embodiment 10. The sample device of any of embodiments 1-9, wherein the first cap comprises a first end portion and a first sealing portion that extends from the first end portion.

Embodiment 11. The sample device of any of embodiments 1-10, wherein the first sealing portion comprises a ring shape and is sized and dimensioned to be securely received by the first open end of the body.

Embodiment 12. The sample device of any of embodiments 1-11, wherein the first end portion comprises a pull tab.

Embodiment 13. The sample device of any of embodiments 1-12, wherein the second cap comprises a second end portion and a second sealing portion that extends from the second end portion.

Embodiment 14. The sample device of any of embodiments 1-13, wherein the second sealing portion comprises a ring shape and is sized and dimensioned to be securely received by the second open end of the body.

Embodiment 15. The sample device of any of embodiments 1-14, wherein the second end portion comprises a pull tab.

Embodiment 16. The sample device of any of embodiments 1-15, wherein the plug comprises a handle portion.

Embodiment 17. The sample device of any of embodiments 1-16, wherein the body is made from a clear plastic.

Embodiment 18. A stand for one or more sample devices, comprising: a base portion; a holder portion comprising at least one holder sized and dimensioned to hold at least one sample device of the one or more sample devices such that the at least one sample device is positioned above the base; and a support portion extending between the base portion and the holder portion.

Embodiment 19. The stand of embodiment 18, wherein the at least one sample device comprises a first coupling component and the holder portion comprises a second coupling component complementary to the first coupling component.

Embodiment 20. The stand of any of embodiments 18-19, wherein the at least one holder is sized and dimensioned to hold the at least one sample device such that the at least one sample device does not contact the base.

Embodiment 21. The stand of any of embodiments 18-20, wherein the at least one holder comprises a C-shaped portion extending about perpendicularly from the support portion.

Embodiment 22. The stand of any of embodiments 18-21, wherein the C-shaped portion comprises flexible end portions.

Embodiment 23. The stand of any of embodiments 18-22, wherein the C-shaped portion comprises a gripping material.

Embodiment 24. The stand of any of embodiments 18-23, wherein the C-shaped portion comprises a compressible material.

Embodiment 25. The stand of any of embodiments 18-24, wherein the C-shaped portion is configured to receive the at least one sample device via a snap fit.

Embodiment 26. The stand of any of embodiments 18-25, wherein the C-shaped portion is adjustable.

Embodiment 27. A kit, comprising: at least one sample device of any of embodiments 1-17; and a stand of any of embodiments 18-26.

Embodiment 28. The kit of embodiment 27, wherein the at least one sample device comprises between 2-25 sample devices.

Embodiment 29. The kit of embodiment 27 or embodiment 28, further comprising a preservative.

Embodiment 30. A kit, comprising: at least one sample device of any of embodiments 1-17; and a preservative.

Embodiment 31. The kit of embodiment 30, further comprising a condom.

Embodiment 32. A method of providing a sample for analysis, comprising: depositing a sample into a condom comprising a top portion and a reservoir portion; placing the condom including the sample in a collection tube having a first end by securing a top portion of the condom around the first end of the collection tube with the reservoir portion positioned within the tube; and securing the top portion of the condom around the first end of the collection tube with a top lid.

Embodiment 33. The method of embodiment 32, wherein the collection tube and top lid are components of a sample device, and wherein the sample device comprises a sample device of any of embodiments 1-17.

Embodiment 34. A method of removing a sample for analysis, comprising: placing a sample device on a stand, the sample device comprising a body having a first end and a first cap coupled to the first end, wherein the sample device holds a condom holding the sample; removing a plug from the top cap to expose an opening, keeping the top cap securely in place coupled to the first end; and inserting a portion of a pipette through opening to collect the sample directly from a reservoir portion of the condom positioned within the tube.

Embodiment 35. The method of embodiment 34, wherein the sample device comprises a sample device of any of embodiments 1-17.

Embodiment 36. The method of embodiment 34 or embodiment 35, wherein the stand comprises a stand of any of embodiments 18-26.

Embodiment 37. The method of any of embodiments 34-36, further comprising: prior to inserting the portion of the pipette; placing a tube of an air delivery device through the opening; and delivering air into a condom secured in the sample device and holding the sample via the air delivery device.

Embodiment 38. The method of any of embodiments 34-37, further comprising: applying the sample to a slide for analysis.

Embodiment 39. A kit for a sample, comprising: a body having a first open end and a second open end; a first cap configured to removably couple to the body at the first open end; and a second cap configured to removably couple to the body at the second open end, wherein at least one of the first and second caps comprises a first opening and a removable plug sized and dimensioned to seal the first opening.

Embodiment 40. The kit of embodiment 39, wherein the first opening is sized and dimensioned for receiving at least a portion of a pipette

Embodiment 41. The kit of any of embodiments 39-40, wherein the first opening is sized and dimensioned for receiving at least a portion of an air pump tube.

Embodiment 42. The kit of any of embodiments 39-41, wherein the first cap comprises a first end portion and a first sealing portion that extends from the first end portion.

Embodiment 43. The kit of any of embodiments 39-42, wherein the first sealing portion comprises a ring shape and is sized and dimensioned to be securely received by the first open end of the body.

Embodiment 44. The kit of any of embodiments 39-43, wherein the first end portion comprises a pull tab.

Embodiment 45. The kit of any of embodiments 39-44, wherein the plug comprises a sealing portion and a handling portion, wherein the first opening has a first diameter, wherein the handling portion has a second diameter that is larger than the first diameter, and wherein the sealing portion has a third diameter that is smaller than the first diameter.

Embodiment 46. The kit of any of embodiments 39-45, wherein the body is made from a clear plastic.

Embodiment 47. The kit of any of embodiments 39-46, further comprising: a packaging containing a preservative suitable for preserving the sample for a period of at least 12 hours.

Embodiment 48. The kit of any of embodiments 39-47, wherein the preservative comprises a curcumin powder.

Embodiment 49. The kit of any of embodiments 39-48, wherein the preservative is suitable for preserving the sample at room temperature for a period of at least 12 hours.

Embodiment 50. The kit of any of embodiments 39-49, further comprising a stand for the sample device.

Embodiment 51. The kit of embodiment 50, wherein the stand comprises: abase portion; a holder portion comprising at least one holder sized and dimensioned to hold at least one sample device of the one or more sample devices such that the at least one sample device is positioned above the base; and a support portion extending between the base portion and the holder portion.

Embodiment 52. The kit of any of embodiments 50-51, wherein the sample device further comprises a first coupling component, and wherein the at least one holder comprises a second coupling component complementary to the first coupling component.

Embodiment 53. The kit of any of embodiments 50-52, wherein at least one of the sample device and the holder comprises a gripping material.

Embodiment 54. The kit of any of embodiments 50-53, wherein the at least one holder is sized and dimensioned to hold the sample device such that the sample device does not contact the base.

Embodiment 55. The kit of any of embodiments 50-54, wherein the at least one holder comprises a C-shaped portion extending about perpendicularly from the support portion.

Embodiment 56. The kit of any of embodiments 50-55, wherein the C-shaped portion comprises flexible end portions.

Embodiment 57. The kit of any of embodiments 50-56, wherein the C-shaped portion comprises a gripping material.

Embodiment 58. The kit of any of embodiments 50-58, wherein the C-shaped portion comprises a compressible material.

Thus, specific examples of devices, kits, and methods for samples have been disclosed. The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

It will be understood that the benefits and advantages described above may relate to one embodiment or may relate to several embodiments. Aspects described in connection with one embodiment are intended to be able to be used with the other embodiments. Any explanation in connection with one embodiment applies to similar features of the other embodiments, and elements of multiple embodiments can be combined to form other embodiments. The embodiments are not limited to those that solve any or all of the stated problems or those that have any or all of the stated benefits and advantages.

The illustrations may include exaggerated dimensions and graphical representation to better illustrate the referenced items shown, and are not considered limiting unless expressly stated as such.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

As used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Also, as used herein, and unless the context dictates otherwise, the term “coupled to” is intended to include both direct coupling (in which two elements that are coupled to each other contact each other) and indirect coupling (in which at least one additional element is located between the two elements).

Reference throughout this specification to “an embodiment” or “an implementation” means that a particular feature, structure, or characteristic described in connection with the embodiment is included in at least one embodiment or implementation. Thus, appearances of the phrases “in an embodiment” or “in some embodiments” in various places throughout this specification are not necessarily all referring to the same embodiment or a single exclusive embodiment. Furthermore, the particular features, structures, or characteristics described herein may be combined in any suitable manner in one or more embodiments or one or more implementations.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Certain numerical values and ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Groupings of alternative elements or embodiments of the disclosure herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

In the specification, reference may be made to the spatial relationships between various components and to the spatial orientation of various aspects of components as the devices are depicted in the attached drawings. However, as will be recognized by those skilled in the art after a complete reading of the present application, the devices, members, devices, etc. described herein may be positioned in any desired orientation. Thus, the use of terms such as “above,” “below,” “upper,” “lower,” “first”, “second” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

All structural and functional equivalents to the components of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A kit for a sample, comprising:
a sample device, comprising:
a body having a first open end and a second open end;
a first cap configured to removably couple to the body at the first open end; and
a second cap configured to removably couple to the body at the second open end, wherein at least one of the first and second caps comprises a first opening and a removable plug sized and dimensioned to seal the first opening.

2. The kit of claim 1, wherein the first opening is sized and dimensioned for receiving at least a portion of a pipette.

3. The kit of claim 1, wherein the first opening is sized and dimensioned for receiving at least a portion of an air pump tube.

4. The kit of claim 1, wherein the first cap comprises a first end portion and a first sealing portion that extends from the first end portion.

5. The kit of claim 4, wherein the first sealing portion comprises a ring shape and is sized and dimensioned to be securely received by the first open end of the body.

6. The kit of claim 4, wherein the first end portion comprises a pull tab.

7. The kit of claim 1, wherein the plug comprises a sealing portion and a handling portion, wherein the first opening has a first diameter, wherein the handling portion has a second diameter that is larger than the first diameter, and wherein the sealing portion has a third diameter that is smaller than the first diameter.

8. The kit of claim 1, wherein the body is made from a clear plastic.

9. The kit of claim 1, further comprising:
a packaging containing a preservative suitable for preserving the sample for a period of at least 12 hours.

10. The kit of claim 9, wherein the preservative comprises a curcumin powder.

11. The kit of claim 9, wherein the preservative is suitable for preserving the sample at room temperature for a period of at least 12 hours.

12. The kit of claim 1, further comprising a stand for the sample device.

13. The kit of claim 12, wherein the stand comprises:
a base portion;
a holder portion comprising at least one holder sized and dimensioned to hold at least one sample device of the one or more sample devices such that the at least one sample device is positioned above the base; and
a support portion extending between the base portion and the holder portion.

14. The kit of claim 13, wherein the sample device further comprises a first coupling component, and wherein the at least one holder comprises a second coupling component complementary to the first coupling component.

15. The kit of claim 13, wherein at least one of the sample device and the holder comprises a gripping material, and wherein the at least one holder is sized and dimensioned to hold the sample device such that the sample device does not contact the base.

16. The kit of claim 13, wherein the at least one holder comprises a C-shaped portion extending about perpendicularly from the support portion.

17. The kit of claim 16, wherein the C-shaped portion comprises at least one of flexible end portions, a gripping material, and a compressible material.

18. A sample device, comprising:
a body having a first open end and a second open end;
a first cap configured to couple to the body at the first open end, wherein the first cap comprises a central opening;
a second cap configured to couple to the body at the second open end; and
a plug configured to seal the central opening.

19. The sample device of claim 18, wherein the first cap comprises a pull tab.

20. A method of providing a sample for analysis, comprising:
depositing a sample into a condom comprising a top portion and a reservoir portion;
placing the condom including the sample in a collection tube having a first end by securing a top portion of the condom around the first end of the collection tube with the reservoir portion positioned within the tube; and
securing the top portion of the condom around the first end of the collection tube with a top lid.
